(19) United States
(12) Patent Application Publication
(10) Pub. No.: US 2017/0143333 A1
(43) Pub. Date: May 25, 2017

(54) SURGICAL APPARATUS, METHOD FOR USING THE SURGICAL APPARATUS AND SUTURE METHOD

(71) Applicant: UNIVERSITÄT HEIDELBERG, Heidelberg (DE)

(72) Inventor: Richard Kroll, Heidelberg (DE)

(21) Appl. No.: 15/300,184

(22) PCT Filed: Mar. 31, 2015
(86) PCT No.: PCT/EP2015/000697

§ 371 Date: Sep. 28, 2016

(30) Foreign Application Priority Data
Apr. 1, 2014 (DE) 10 2014 004 772.5

(51) Int. Cl. A61B 17/04; A61B 17/06

(52) U.S. Cl. CPC A61B 17/0483; A61B 17/06166; A61B 17/0469; A61B 2017/06176

(57) ABSTRACT

A surgical apparatus for suturing tissue using a surgical suture material having one or more hooking elements is provided. The surgical apparatus includes a suture material transportation member adapted to transport the suture material, a suture material dispensing member adapted to displace the suture material transportation member in a first direction for dispensing suture material to the tissue to be sutured and adapted to apply a specified pull force to the suture material, and a suture material application member adapted to apply the suture material to the tissue so that at least the hooking elements are introduced into the tissue.

### CROSS REFERENCE TO RELATED APPLICATIONS

This application is a National Stage Entry of PCT/EP2015/000697 filed Mar. 31, 2015, which claims priority to from DE Patent Application No. 10 2014 004 772.5 filed Apr. 1, 2014, both of which are incorporated herein in their entirety.

### BACKGROUND

The present application refers to a surgical apparatus, a method for using the surgical apparatus and a suture material.

Laparoscopic or thoracoscopic surgeries usually require suture techniques in which two working channels are provided at the same time in order to fix the suture. The possibility of maneuvering in a laparoscopic or thoracoscopic surgery at e.g. a baby, an urinary bladder, a rectum, a epigastrium, or in a single port surgery is however limited, so that the variety of suture techniques in which two working channels are provided (e.g. bimanuel knotting, suturing according to U.S. Pat. No. 5,728,107A, technique according to Lahodny) is limited.

Laparoscopic tackers, which are known from the prior art (U.S. Pat. No. 7,914,551 B2, U.S. Pat. No. 5,634,584 A, WO 00/57796 A1), are not able on their own to apply a pull force to the surgical material or the body tissue, when fixing a surgical material or a body tissue portion to another surgical material or body tissue portion but require assistance by a further instrument.

The present disclosure is therefore directed to enabling the surgeon to define the distance and tension between tissue portions to be connected with one single instrument, using one single working channel.

Conventional, bimanual knotting in thoracoscopic or laparoscopic surgery requires intensive training of the surgeon. In line with that, absolute time for suturing is longer than in open surgery. Though the technique of extracorporeal knotting is easier to be handled and even applicable for narrow operation sites as the above mentioned ones, it is still highly time consuming.

Hence the present disclosure is also directed to providing a suturing technique that is easy to be learned and to be performed in a time-saving manner.

### BRIEF DESCRIPTION

According to a first aspect a surgical apparatus for suturing tissue by means of a surgical suture material having one or more hooking elements or barbs is provided. The surgical apparatus includes a suture material transportation member being adapted to transport suture material, a suture material dispensing member being adapted to displace the suture material transportation member in a first direction for dispensing suture material to the tissue to be sutured and being adapted to apply a specified pull force (specifically in a second direction different from the first direction, the second direction particularly being substantially opposite to the first direction) on the suture material, and a suture material application member being adapted to apply the suture material to the tissue so that at least the hooking elements are introduced into the tissue.

Accordingly, the surgical apparatus of the present disclosure provides a single apparatus which on the one side dispenses a suture material to the tissue portions to be connected and on the other side provides an apparatus which enables the surgeon to apply a specified pull force to the suture material. The specified (predetermined/predeterminable) pull force enables the surgeon to determine the distance between the tissue portions to be connected after the tissue portions have approached. Specifically, the user such as the surgeon may apply the specified (predetermined or predeterminable) pull force without having to manually displace the surgical apparatus itself, but rather e.g. by (directly and/or indirectly) actuating the suture material dispensing member and/or driving the suture material transportation member. Accordingly, it is advantageously possible to reliably apply the specified pull force particularly without extensive training of the operator.

When transporting the suture material towards the tissue portions, the suture material passes the suture material application member which is adapted to press the hooking elements into the tissue. In other words, the suture material is only laid onto the tissue portions to be connected and the hooking elements are pressed into the tissue to be connected by means of the suture material application member.

As soon as the hooking elements are pressed into a first tissue portion of the tissue portions to be connected, the surgical apparatus is moved toward the second tissue portion. Thereby, the suture material dispensing member is driven in the first direction so that further suture material is dispensed. By defining the amount of the further suture material, which is dispensed, a specified pull force is applied to the suture material specifically in the second direction different from the first direction (particularly substantially opposite to the first direction).

Accordingly, the term “specified” regarding the pull force defines the amount of the pull force which is required to pull the tissue portions together such that a desired distance between the tissue portions remains.

After the specified pull force is applied to the suture material, the surgical apparatus is pressed onto the second tissue portion, so that hooking elements are introduced into the tissue.

In consequence, the surgical apparatus of the present disclosure provides a single apparatus which enables the surgeon to carry out a surgery in a limited space. In particular, the surgical apparatus provides the possibility to perform surgeries requiring only one working channel in which suture material may be dispensed towards the tissue portions to be connected and in which the surgeon may adjust the distance between the tissue portions to be connected in a simple manner by applying a specified pull force to the suture material.

The suture material may be adhered onto the suture material transportation member in order to be transported.

The suture material is put or arranged on the suture material transportation member for being transported by the same. Specifically, the suture material transportation member at least partly is provided with an adhesion promoting member (such as a surface coating) to adhere the suture material onto the suture material transportation member. Adhesion specifically may be provided by mechanical adhesion, chemical adhesion (e.g. by ionic bonding, covalent bonding and/or hydrogen bonding), dispersive adhesion or physisorption, electrostatic adhesion and/or diffusive adhesion between the suture material and the adhesion promoting member. Further specifically, the suture material transportation member is provided with (particularly covered by) a glue or adhesion promoting surface so that the suture material is fixed substantially by adhesion to or (temporarily) held by adhesion on the suture material transportation member. The glue may be a pressure sensitive adhesive. Thereby, the suture material can be reliably held on and transported by the suture material transportation member even when the surgical apparatus is moved by the surgeon. Specifically, the adhesion forces between the suture transportation member and the suture material are set to such an amount that (i) the suture material can be appropriately dispensed and/or (ii) the pull forces on the patient's tissue are appropriately exerted to such a degree that the tissue portions is pulled together to set or maintain a desired distance between the tissue portions.

The suture material dispensing member may be further adapted to displace the suture material transportation member in a second direction.

Thereby, the surgeon is enabled to correct the amount of dispensed suture material. If the surgeon dispenses too much suture material, which might result in a too low pull force, the suture material transportation member could be displaced in a second direction different from the first direction, which particularly is substantially opposite to the first direction. Thereby, the length of the dispensed suture material can be reduced and the pull force increased.

In one embodiment, the surgical apparatus further includes a suture material separation member being adapted to separate the suture material from the suture material transportation member.

A suture material cutting member may be adapted to cut the suture material in an area where the suture material is separated from the suture material transportation member by the suture material separation member.

As the suture material transportation member is separated from the suture material in an area, a suture material cutting member is able to cut the suture material in a simple manner without affecting the suture material transportation member.

As soon as the specified (predetermined/predeterminable) pull force is applied to the suture material, the surgeon can use or activate the suture material cutting member in order to cut the suture material.

In one embodiment, the suture material transportation member is a conveyor belt and/or a conveyor chain and/or a conveyor slide member.

The suture material transportation member can include only a conveyor belt or a conveyor chain or a conveyor slide member. Alternatively, the suture material transportation member includes in parts a conveyor belt and in parts a conveyor chain, which are connected to each other. Alternatively, the suture material transportation member includes in parts a conveyor belt and in parts a conveyor slide member, which are connected to each other. Alternatively, the suture material transportation member includes in parts a conveyor chain and in parts a conveyor slide member, which are connected to each other.

The suture material separation member may include at least one guide roller.

The at least one guide roller is arranged at a position displaced with respect to the level at which the suture material is transported. The suture material transportation member is guided around the at least one guide roller so that the suture material transportation member is guided away from the transportation level of the suture material.

The at least one guide roller provides a simple and space-saving construction, while the suture material cutting member still has sufficient space for cutting the suture material.

In one embodiment, the suture material cutting member comprises at least one knife element or a heating wire.

The knife element may be arranged next to the suture material to be cut. When the surgeon moves the knife element, the knife element may head towards a slat surface which is arranged at an opposite side of the suture material. By pressing the knife element onto the slat surface, the suture material may be cut.

Alternatively, the surgical apparatus includes two knife elements which are arranged separately from each other adjacent the suture material to be cut. When the surgeon activates the knife elements, the knife elements approach so that the suture material is cut.

Further alternatively, the suture material cutting member may have a heating wire which is arranged adjacent to the suture material. When the surgeon activates the heating wire, the suture material is cut by means of the heat of the glow wire. The heating wire may contact the suture material.

According to a second aspect, a method for using a surgical apparatus is provided. The method includes providing a surgical apparatus according to any one of the above described surgical apparatus, driving the suture material transportation member in the first direction by means of the suture material dispensing member so that the suture material is transported to the suture material application member and so that a specified pull force is applied to the suture material, and introducing at least the hooking elements of the suture material into the tissue by means of the suture material application member.

The method may include the further step of driving the suture material transportation member in the second direction by means of the suture material dispensing member.

Further, the suture material may be transported by adhering the suture material onto the suture material transportation member.

Further, the method further includes a step of separating the suture material from the suture material transportation member by the suture material separation member.

Further, the method includes the step of cutting the suture material at a position where the suture material is separated from the suture material transportation member by the suture material separation member.

According to a third aspect, a suture material is provided. The suture material includes an elongated body, a plurality of barbs attached to the elongated body, at least one auxiliary barb which is attached to at least one of barbs, wherein the auxiliary barb is directly or indirectly attached to the barb such that the auxiliary barb is abutted on the barb when the barb is inserted into the tissue and that the auxiliary barb projects from the barb when a pull force is applied to the barb in a direction opposite to an insertion direction of the barb.

The pull force may be applied to the barb by the surgical apparatus as described above or by the natural movement of the tissue.

Accordingly, the suture material of the fourth aspect may be advantageously used together with the surgical apparatus as described above. By means of the suture material application member of the surgical apparatus the barbs are inserted or pressed into the tissue, while the elongated body of the suture material does not need to be inserted. The elongated body rather lies on the top surface of the tissue. Although only the barbs are inserted into the tissue, the barbs are prevented from being pulled out of the tissue as the auxiliary barbs project from the barbs and block an outwards movement of the barbs.

In one embodiment, the barbs are attached perpendicular to the elongated body.

A perpendicular arrangement of the barbs with respect to the elongated body may be advantageous, as the barbs are able to be easily inserted into the tissue when using a surgical apparatus as explained above.

In one embodiment, a deflection stopping member is formed at at least one of the barbs which is adapted to stop a deflection of the at least one auxiliary barb at a specified angle.

The deflection stopping member may be uniformly formed with the barb or may be affixed to the barb. The deflection stopping member may be attached to the barb at a position so that a deflection angle of the auxiliary barb is limited.

When a pull force is applied to the barb, the auxiliary barb moves from an abutted position to a projection position. If the pull force is too strong, there is a risk that the deflection angle between the auxiliary barb and the barb becomes too large so that the auxiliary barb is no longer able to block a removal of the barb.

The deflection stopping member may stop the deflection angle of the auxiliary barb at an angle between about 40° and about 90°, particularly between about 65° and 85° and more particularly between about 70° and 80°.

In one embodiment, the deflection stopping member provides a deflection stopping surface against which the auxiliary barb is pressed if a too high pull force is applied to the barb. Thereby, a too strong deflection of the auxiliary barb can be prevented.

Further, there is disclosed a method for suturing a tissue. The method includes providing a surgical apparatus according to any one of the above described surgical apparatus, driving the suture material transportation member in the first direction by means of the suture material dispensing member so that the suture material is transported to the suture material application member and so that a specified pull force is applied to the suture material, and introducing at least the hooking elements of the suture material into the tissue by means of the suture material application member.

### BRIEF DESCRIPTION OF THE DRAWINGS

The features and advantages of the present disclosure become clearer when studying the following detailed description of exemplary embodiments and the accompanied figures. It is obvious that, although embodiments are separately described, single features of these embodiments can be combined to additional embodiments.

FIG. 1 discloses a section view of a surgical apparatus according to a first embodiment;

FIG. 2 discloses a section view of a surgical apparatus according to a second embodiment.

FIG. 3 discloses a section view of a surgical apparatus according to a third embodiment.

FIG. 4 discloses an extract of a suture material.

FIG. 5A discloses a schematic drawing of a staple in the tensioned position, which comprises a water-soluble dry substance.

FIG. 5B discloses a schematic drawing of the staple of FIG. 5A in the closed position.

FIG. 6A discloses a schematic drawing of a staple in the open position, which comprises a ductile intermediate portion.

FIG. 6B discloses a schematic drawing of the staple of FIG. 6A in the closed position.

### DETAILED DESCRIPTION

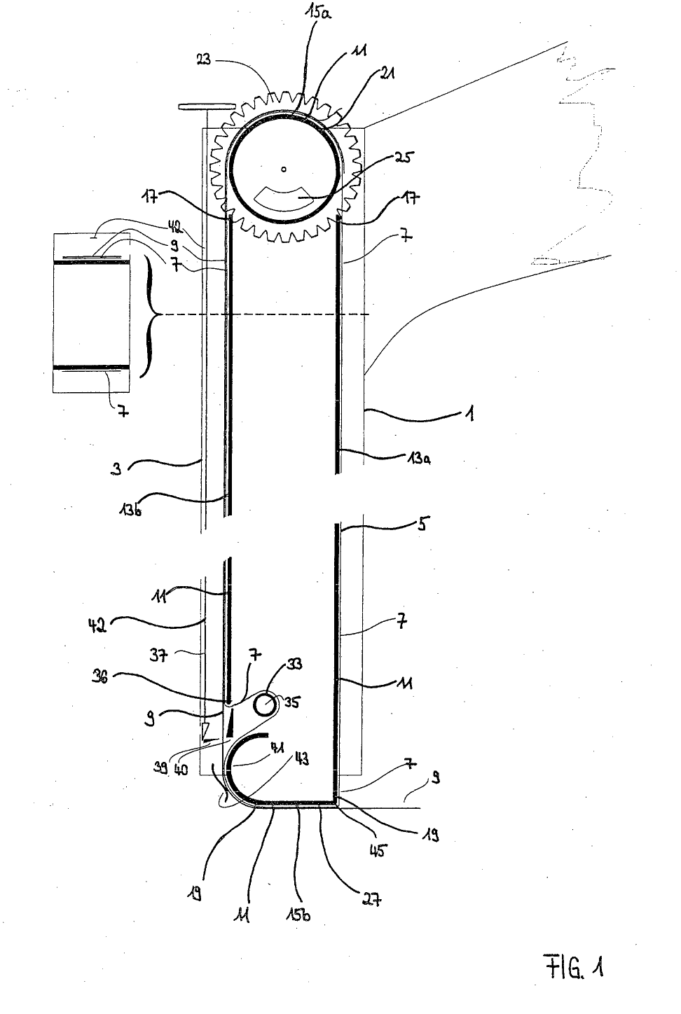

FIG. 1 discloses a section view of a surgical apparatus 1 according to a first embodiment.

The surgical apparatus 1 includes a housing 3 in which a suture material transportation member 5 is housed. The suture material transportation member 5 includes a conveyor belt 7, on which suture material 9 is transportable, and a supporting member 11. The conveyor belt 7 may be endless. Specifically, the suture material 9 is transported by the suture material transportation member 5 by being (at least temporarily) adhered thereto. Accordingly, it is possible to transport and displace the suture material 9 particularly without having to prior anchor it into the patient's tissue in view of displacing it. Furthermore, since the suture material 9 can be dispensed and/or displaced by means of the suture material transportation member 5 it is not necessary to first fix it to the patient's tissue in order to be dispensed or drawn out from the housing 3. This is particularly advantageous when the patient's tissue being sutured is specifically brittle or soft and which may, thus, be damaged by an unnecessary force (such as a pulling action) acting on the suture material 9. Additionally, by dispensing the suture material 9 under the assistance of the suture material transportation member 5 a surface anchoring or fixing of the suture material 9 on the patient's tissue is promoted.

Particularly, the conveyor belt 7 is at least partly covered with an adhesion promoting surface (such as a glue, pressure sensitive adhesive or the like) to provide appropriate adhesion of the suture material 9 to the suture material transportation member 5 via the conveyor belt 7. Accordingly, the suture material 9 can be properly dispensed from the housing 3 by reliably displacing it by means of the suture material transportation member 5 on which it at least partly adheres. Alternatively or additionally, the suture material transportation member 5 may comprise a conveyor belt and/or a (not shown) conveyor chain and/or a (not shown) conveyor slide member. Specifically, the conveyor belt and/or the conveyor slide may be at least partly covered with the adhesion promoting surface (such as a glue, pressure sensitive adhesive or the like) to provide appropriate adhesion of the suture material 9 to the suture material transportation member 5 via the conveyor belt and/or the conveyor slide.

The conveyor belt 7 is guided on an outer surface of the supporting member 11. The supporting member 11 comprises at least two longitudinal supporting elements 13 and at least two redirecting supporting member 15.

The longitudinal supporting elements 13 are arranged in parallel and separated from each other. Adjacent to first ends 17 of the longitudinal supporting elements 13 a first redirecting supporting element 15a is arranged, which is formed as a roller. The first redirecting supporting element 15a may have a diameter which is identical to the distance between the two longitudinal supporting elements 13. By means of the first redirecting supporting element 15a, the conveyor belt 7 is guideable such that the conveyor belt 7 moves along the outer surface of a first longitudinal supporting element 13a, around the first redirecting supporting element 15a to the outer surface of the second longitudinal supporting element 13b.

Second ends 19 of the longitudinal supporting elements 13 are connected by means of a second redirecting supporting element 15b. The second redirecting supporting element 15b may be arranged perpendicular to the longitudinal supporting elements 13. The conveyor belt 7 is guided along the outer surface of the second redirecting supporting element 15 such that the conveyor belt 7 is guided from the second longitudinal supporting element 15b to the first longitudinal supporting element 15a.

The respective outer surface of the longitudinal supporting elements 13 is the surface which is opposite to the surface facing the other longitudinal supporting element 13. The outer surface of the second redirecting supporting element 15b is the surface which is opposite to the surface facing the first redirecting supporting element 15a. The outer surface of the first redirecting supporting element 15a is the lateral surface of the roller.

As mentioned above, on the outer surface of the conveyor belt 7, the suture material 9 is arrangeable in order to be transported or displaced by the conveyor belt 7. The conveyor belt 7 may provide adhesive material (as a particular adhesion promoting surface) on the outer surface of the conveyor belt 7 so that the suture material (temporarily) sticks or adheres to the conveyor belt 7. Accordingly, the suture material 9 is prevented from falling off the conveyor belt 7 and can be safely conveyed.

According to the first embodiment, the suture material transportation member is manually driven.

A suture material dispensing member 21 is fixedly attached to the first redirecting supporting member 15a. The suture material dispensing member 21 is formed as a disc 23 and the rotation axis of the disc 23 is identical to the rotation axis of the first redirecting supporting member 15a. Via a disc opening (not shown) in the housing 3 an operator such as a surgeon is able to move the disc 23 in a first operation direction and also in a second operation direction (different from the first operation direction, particularly substantially opposite to the first operation direction) in order to displace the conveyor belt 7. The disc 23 may project partly beyond the housing 3 in order to simplify the manipulation by the surgeon. In other words, the operator such as the surgeon can dispense the suture material 9 in a dispensing direction (as a particular first direction), in order to be able to apply the suture material 9 to the patient's tissue, and subsequently displace the suture material 9 in a retracting direction (as a particular second direction) different from the dispensing direction (particularly substantially opposite thereto) in order to apply a specified (predetermined or predeterminable) pull force on at least part of the patient's tissue.

A braking member 25 may be attached to the disc 23, which is adapted to allow a movement of the disc 23 only by applying a predetermined force. The braking member 25 may be a braking shoe.

When moving the disc 23 in the first direction, the conveyor belt 7 is moved in the first direction so that the suture material 9 is transported towards the tissue (not shown) to be connected.

In particular, the suture material 9 is transported towards the second redirecting supporting member 15b. The second redirecting supporting member 15b is accessible from the outer side of the housing 3 via a housing opening (not shown). The second ends 19 of the longitudinal supporting elements 13 may project towards the outer side of the housing 3 so that the second redirecting supporting member 15b is arranged outside the housing 3.

The outer surface of the second redirecting supporting element 15b is a suture material application member 27.

The surgeon, when using the surgical apparatus 1, puts and/or presses the outer surface of the second redirecting supporting element 15b on the tissue to be connected, so that the suture material 9 which is presently located on the conveyor belt 7 in the area of the second redirecting supporting element 15b is at least partly introduced into the tissue. Specifically, a surface connection (or an anchoring over a discrete surface area) is advantageously achieved between the suture material 9 and the patient's tissue.

The suture material 9, which is transported by the conveyor belt 7 and dispensed to the tissue, may include an elongated body 67 and hooking elements. The hooking elements may be barbs 31 or staples 29, which are attached to the elongated body 67. Accordingly, when putting the second redirecting supporting element 15b on the tissue to be connected, the barbs 31 or the staples 29 are introduced into the tissue.

First of all the second redirecting supporting element 15b is put on a part of a first tissue portion so that the hooking elements are introduced into the first tissue portion.

Then, in order to pull the first tissue portion and a second tissue portion together, the surgeon operates (e.g. turns) the suture material dispensing member 21 in a first direction for dispensing further suture material 9 in a specified (predetermined or predeterminable) amount when moving the surgical apparatus 1 from the first tissue portion to the second tissue portion. Moreover, by exerting a (braking) force or by operating (e.g. turning) the suture material dispensing member 21 in a second direction, which particularly is a direction substantially opposite to the dispensing direction, a specified (predetermined or predeterminable) pull force is applied to the first tissue portion via the suture material 9, whereby the first tissue portion is pulled towards the second tissue portions in a specified (predetermined or predeterminable) amount. In other words, it is possible to apply a pull force to the suture material 9 in a selectable amount, which particularly can be freely determined or adjusted by the operator such as the surgeon e.g. depending on actual operative conditions and/or the intended suture. Then, the second redirecting supporting element 15b is put on a part of the second tissue portion so that the hooking elements are introduced into the second tissue portion. The extent of tightening the tissue portions is defined by the amount of the pull force applied by the suture material dispensing portion 21.

Accordingly, the surgical apparatus 1 provides a single apparatus by which the surgeon is able to define the extent of tightening of the tissue portions to be connected and which requires only one working channel. Moreover, the surgical apparatus is easy to handle and does not require extensive experiences from the operator such as the surgeon.

Additionally, the surgical apparatus 1 provides a suture material separation member 33, which is adapted to separate the conveyor belt 7 from the suture material 9.

The second longitudinal supporting element 13b specifically is interrupted in a portion. In this portion, a guide roller 35 is located displaced from the level of the second longitudinal supporting element 13b. In particular, the guide roller 35 is displaced to an inner side of the second longitudinal supporting element 13b. The “inner side” is defined as the side of the second longitudinal supporting element 13b which faces the first longitudinal supporting element 13a.

The conveyor belt 7 at least partly is guided around the guide roller 35 in the portion where the second longitudinal supporting element 13b is interrupted. The conveyor belt 7 may be deflected towards the guide roller 35 by a free end 36 of the second longitudinal supporting element 13. The free end 36 of the second longitudinal supporting element 13 faces the interrupted portion of the second longitudinal supporting element 13. By means of the guide roller 35 the conveyor belt 7 is guided towards an inner side of the second longitudinal supporting element 13b. The suture material 9, however, is not guided to the inner side but remains in its original level. Accordingly, the suture material 9 is exposed in the portion where the second longitudinal supporting element 13b is interrupted.

The exposed portion of the suture material 9 is advantageous for cutting the suture material 9 at this position.

Therefore, the surgical apparatus 1 includes a suture material cutting member 37, which is arranged in the portion where the second longitudinal supporting element 13b is interrupted.

As disclosed in FIG. 1, the suture material cutting member 37 may comprise a knife element 39 and a slat surface 40. The knife element 39 and the slat surface 40 are arranged adjacent to the suture material 9 in the portion, where the second longitudinal supporting element 13b is interrupted, particularly at substantially opposite sides. The knife element 39 is connected to an actuation member 42, which may be operated by the surgeon. When operating the actuation member 42, the knife element 39 heads towards the slat surface 40. By pressing the knife element 39 onto the slat surface 40, the suture material 9 is cut.

The slat surface 40 can optionally be attached to a rocker (not shown) or a leaf spring (not shown), so that the slat surface 40 may move around an axis which is orthogonally oriented in relation to the knife element 39.

The actuation member 42 may be a lever which projects to the outside of the housing 3. By pulling or pushing the lever the knife element 39 is moved as explained above.

In order to guide the conveyor belt 7 smoothly back to the outer surface of the second longitudinal supporting element 13b in the first direction after the conveyor belt 7 has passed the guide roller 35, a reassembling portion 41 is formed in the second longitudinal supporting element 13b. The reassembling portion 41 is connected to the second redirecting supporting element 15b and is formed in a rounded shape. The reassembling portion 41 may be formed as a semicircle, which is open towards the inner side of the second longitudinal supporting element 13b.

A leaf spring 43 may be arranged adjacent an outer side of the reassembling portion 41, which is adapted to press the suture material 9 back against the conveyor belt 7. Alternatively, a plurality of brushes (not shown) may be arranged in a row adjacent the outer side of the reassembling portion 41.

The step of cutting the suture material 9 is may be carried out after the hooking elements are pressed into both tissue portions by the second redirecting supporting element 15b.

As shown in FIG. 1, the second redirecting supporting element 15b and the first longitudinal supporting element 13a are arranged perpendicular to each other. At the edge 45 built by these elements the suture material 9 and the conveyor belt 7 are separated.

In case the used suture material 9 is longer than the length of the conveyor belt 7, the surgical apparatus 1 may provide a storing member (not shown), where the suture material 9 is stored and from which suture material 9 is dispensed to the conveyor belt 7.

Further, a flushing channel (not shown) may be attached to the second redirecting supporting element 15b. The flushing channel may be used if the surgical apparatus 1 is used with suture material 9 having a plurality of staples 29.

Moreover, the first longitudinal supporting element 13a and/or the second longitudinal supporting element 13b may be provided with one or more pressure exerting elements (not shown) being adapted to exert a pressure on the suture material 9 arranged on the suture material transportation member 5. The one or more pressure exerting elements may be in the form of or comprise one or more suction ducts which apply a force to the suture material 9 by means of a suction (i.e. pressure smaller than about 1 bar). Alternatively or additionally the one or more pressure exerting elements may be in the form of or comprise one or more pressure ducts for applying a (positive) pressure (i.e. pressure greater than about 1 bar) onto the suture material 9. The suction duct(s) and/or the pressure duct(s) particularly may be part of or integrated in the suture material application member 27. The provision of the one or more pressure exerting elements advantageously permits to apply or exert a suitable contact pressure (specifically an under-pressure and/or over-pressure) on the suture material 9 being transported (specifically on the conveyor belt 7 of) the suture material transportation member 5. Specifically, it is possible to use the flushing channel described above to also integrate or form the suction duct(s) and/or the pressure duct(s).

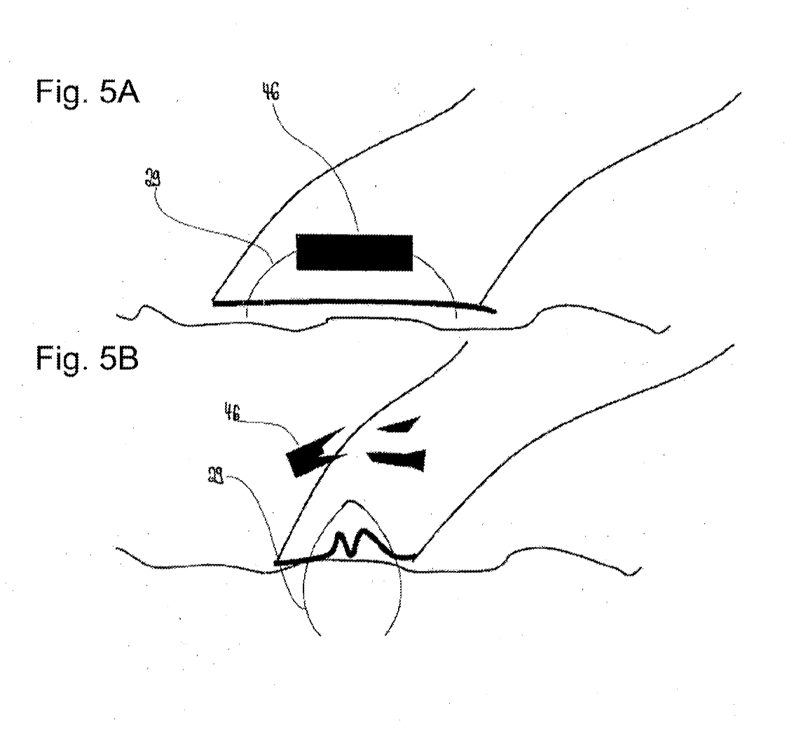

As shown in FIGS. 5A and 5B, each of the staples 29 may have an blocking device 46 by which the respective staple 29 is held in a tensioned (open) position. The blocking device 46 is formed of a highly water-soluble dry substance. By flushing the blocking device 46 of a staple 29 by means of the flushing channel arranged at the second redirecting supporting element 15b, the blocking device 46 is solved. Thereby, the staple 29 leaves the tensioned position (FIG. 5A) and moves to the closed position (FIG. 5B) so that staple 29 may be hooked in the tissue.

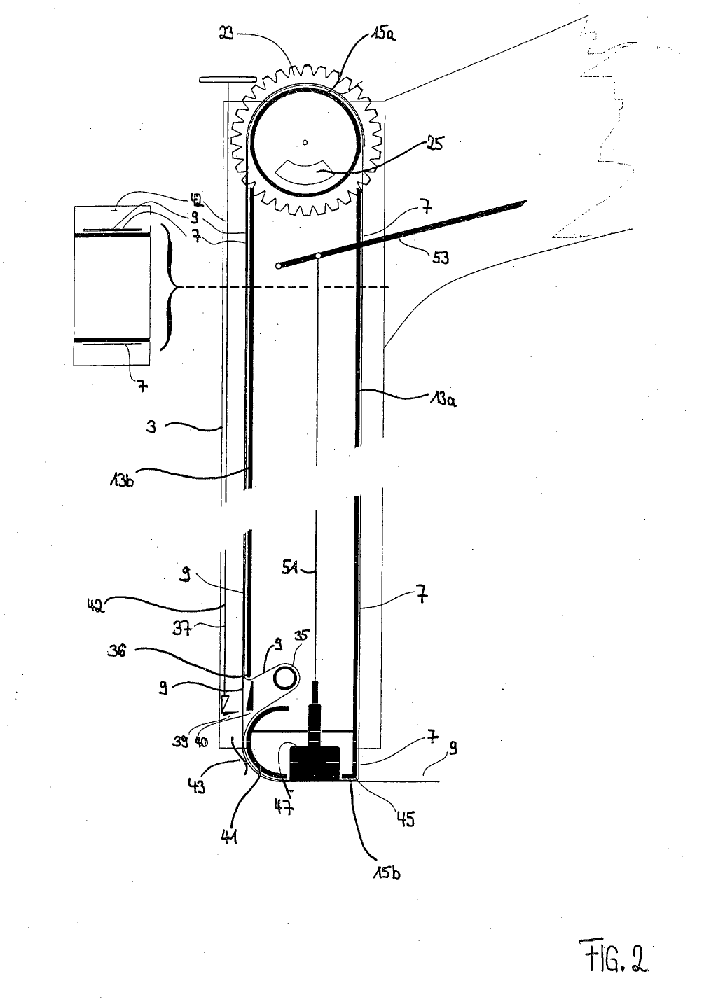

FIG. 2 discloses a section view of a second embodiment.

The surgical apparatus 1 of the second embodiment provides additionally a suture material squeezing member 47. Any other elements of the surgical apparatus 1 of the second embodiment are similar or identical to the first embodiment so that the description of the first embodiment also applies for the second embodiment.

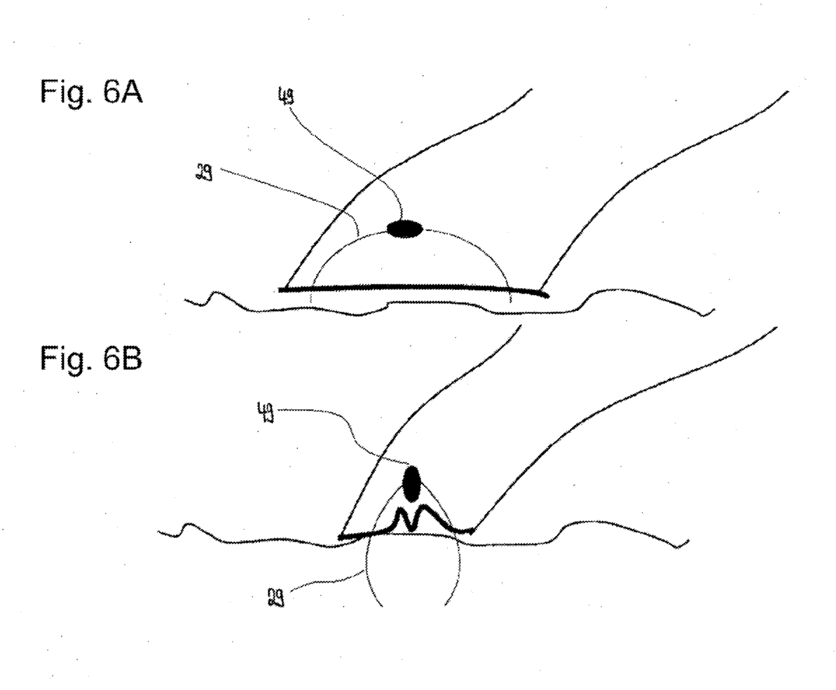

The second redirecting supporting element 15b is interrupted in a portion. In this portion a suture material squeezing member 47 is arranged. The suture material squeezing member 47 includes tongs, which are movable between an open position and a closed position. When moving the tongs from the open position towards the closed position, the tongs squeeze a staple 29 of the suture material 9. Thereby, the staple 29 is hooked in the tissue. For bending the staple 29, the staple 29 may include a ductile intermediate portion 49 as shown in FIGS. 6A and 6B. FIG. 6A discloses the staple 29 having the intermediate portion 49 in an open position, while FIG. 6B discloses the same staple 29 in the closed (squeezed) position.

The tongs may be connected to a push rod 51, which is attached rotatably to a pressing member handle 53. The pressing member handle 53 extends to the outside of the housing 3 so that the surgeon is able to move the tongs and also able to operate the tongs.

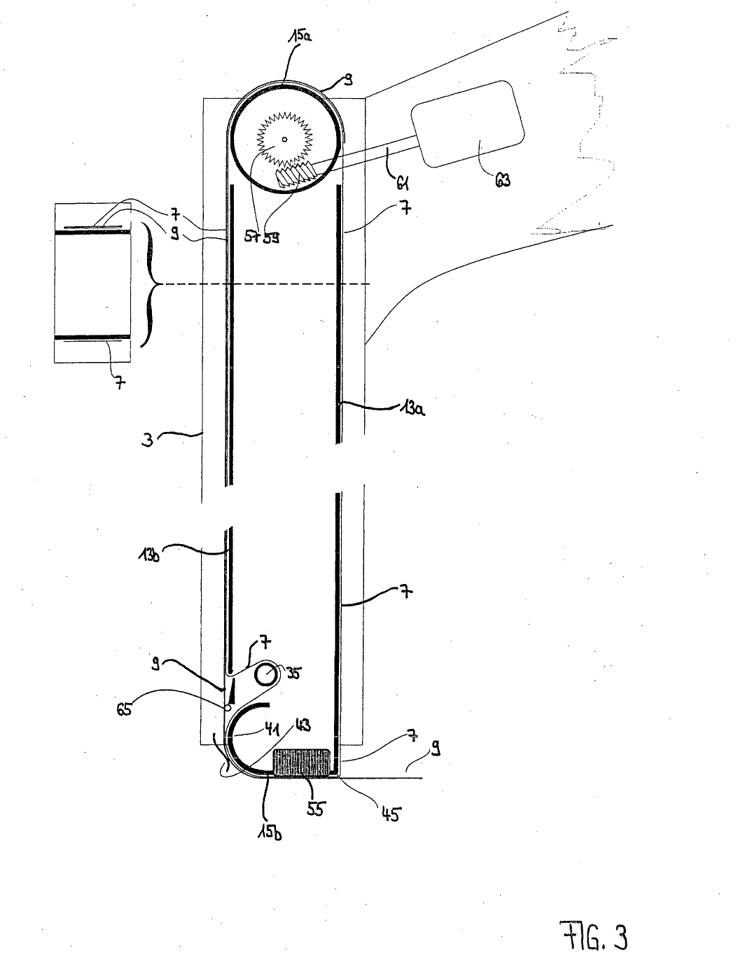

FIG. 3 discloses a section view of a surgical apparatus 1 according to a third embodiment.

The surgical apparatus 1 according to the third embodiment differs from the first embodiment in that the suture material dispensing member is electrically driven. Optionally, the suture material cutting member is differently formed and may be a thermoelement 55 is attached to the second redirecting supporting element 15b. Any other elements of the surgical apparatus 1 of the third embodiment are similar or identical to the first embodiment so that the description of the first embodiment also applies for the second embodiment.

In particular, the third embodiment does not provide a disc 23 which is attached to the first redirecting supporting element 15a. In the third embodiment, a gear ring 57 is attached to the first redirecting supporting element 15a such that the rotation axis of the gear ring 57 is identical to the rotation axis of the first redirecting supporting element 15a. The gear ring 57 engages with a screw drive element 59. The screw drive element 59 is attached to a rotatable rod 61, which is connected to an electromotor 63. The electromotor 63 is adapted to rotate the rod 61 and thereby the screw drive element 59. By rotating the screw drive element 59 the first redirecting supporting element 15a is rotated so that the conveyor belt 7 is displaceable.

Optionally, the knife element 39 of the first and second embodiments may be replaced by a heating wire 65. The heating wire 65 is arranged adjacent to the suture material 9 in the portion where the second longitudinal supporting element 13b is interrupted. The heating wire 65 may contact the suture material 9. By activating the heating wire 65, the suture material may be cut.

Further optionally, a thermoelement 55 is attached to the second redirecting supporting element 15b. If a suture material 9 with staples 29 having an intermediate portion 49 as shown in FIGS. 6A and 6B is used, the staples 29 may be bent from the open position to the closed position by applying heat to the staples 29 by means of the thermoelement 55.

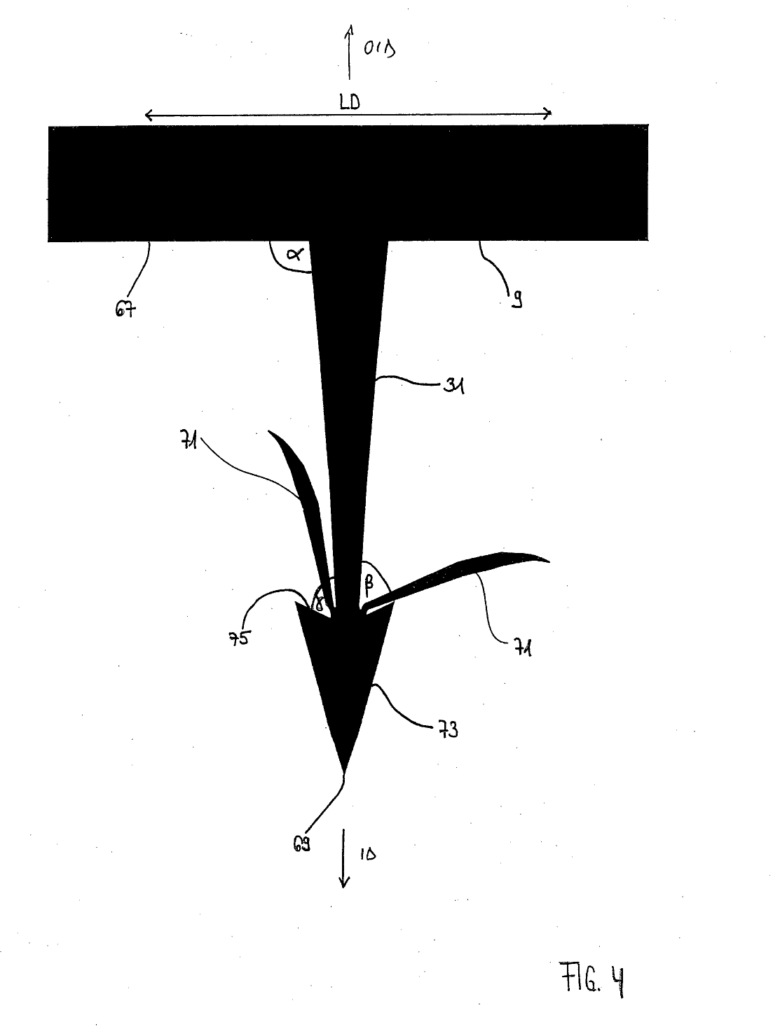

FIG. 4 shows an extract of a suture material 9, which may be used in combination with any one of the above described embodiments of a surgical apparatus 1.

The suture material 9 includes an elongated body 67 which extends in a longitudinal direction LD. The elongated body 67 may have a circular cross-section, when the elongated body 67 is cut in a direction perpendicular to the longitudinal direction LD. However, any other shapes like oval or triangular are possible. The elongated body 67 may have a diameter between about 1 mm and about 200 mm, particularly between about 2 mm and about 8 mm and more particularly between about 3 mm and about 7 mm.

Further, the elongated body 67 may have a length between about 10 mm to about 700 mm, particularly between about 100 mm and about 500 mm and more particularly between about 200 mm and about 300 mm.

The suture material 9 is made of an absorbable material and/or a non-absorbable material. In particular, biologic or synthetic polymers may be used as a suture material 9. Exemplary non-absorbable materials are polyamide (e.g. Nylon), polypropylene, silk, cotton, caprolactam, polyester, polybutester, polyvinyl alcohol. Examplary absorable materials are polyglycol acid, polylactic acid, polycaprolactone, polydioxanone, collagen. Alternatively, a combination of absorbable and non-absorbable material are used for a suture material 9. Exemplary, a combination of Nylon and polylactic acid may be used as a suture material. Moreover, the suture material 9 is flexibly formed.

One or more barbs 31 are attached to the elongated body 67. FIG. 4 shows only one barb 31 of these barbs 31. The barb 31 may form a barb angle α together with the elongated body 67 between about 60° and about 120°, particularly between about 70° and about 100° and more particularly between about 80° and about 95°. The barb angle α is measured between the elongated body 67 and a line parallel to the insertion direction ID of the barb 31.

The barbs 31 may be uniformly formed with the elongated body 67. Alternatively, the barbs 31 are separately formed and affixed to the elongated body 67.

As shown in FIG. 4, the barb 31 has a tapered shape, which tapers from the elongated body 67 towards the barb point 69. However, the barb 31 may alternatively have a rectangular shape in cross-section.

The barbs 31 may be displaced with respect to each other along the outer surface of the elongated body 67. In particular, the barbs 31 may be arranged in one or more lines, in a spiral arrangement or in circles. The barbs 31 may be arbitrarily displaced along the outer surface of the elongated body 67.

Additionally, at least one auxiliary barb 71 is directly or indirectly attached to at least one of the barbs 31. The auxiliary barb 71 may be uniformly formed with the respective barb 31 or separately formed and affixed to the respective barb 31. The auxiliary barb 71 may be cut into the barb 31.

The auxiliary barb 71 is movable between an abutted position and a projecting position.

The auxiliary barb 71 may be tapered or may be leaf-shaped. As the auxiliary barb 71 abuts on the respective barb 31 to which it is attached in the abutted position when barb 31 is inserted into the tissue, the leaf-shape is advantageous.

If a pull force is applied to the barb 31 in a direction OID opposite to the insertion direction ID of the barb 31, the auxiliary barb 71 no longer abuts on the barb 31 but projects from the barb 31. The auxiliary barb angle β between the auxiliary barb 71 and the barb 31 is thereby defined by the amount of the pull force, the strength of the tissue, and the thickness and material of the auxiliary barb 71.

The auxiliary barb 71 is in the abutted position when the barb 31 is inserted into the tissue so that the auxiliary barb 71 does not obstruct the insertion of the barb 31. The auxiliary barb angle β between the barb 31 and the auxiliary barb 71 may be 0°. Auxiliary barb angles β between about 0° and about 15° refer however also to the abutted position of the auxiliary barb 71. The auxiliary barb 71 on the left side in FIG. 4 shows the abutted position. The auxiliary barb angle β is measured between a line being parallel to the insertion direction ID of the barb 31 and the auxiliary barb 71.

The auxiliary barb 71 on the right side in FIG. 4 is in the projecting position. The auxiliary barb 71 is pressed towards the projecting position, in case a pull force is applied to the barb 31. Such a pull force may be applied to the barb 31 by the surgical apparatus 1 as explained above. Additionally or alternatively a pull force may be applied to the barb 31 even after the surgery by the usual movement of the tissue.

If a pull force is applied to the barb 31, the barb 31 tends to move out of the tissue. In this case the auxiliary barb 71 is however pushed from the abutted position towards the projecting position by the strength of the tissue so that the auxiliary barb 71 is hooked into the tissue. Accordingly, a movement of the barb 31 in a direction opposite to the insertion direction of the barb 31 is prevented.

An auxiliary barb angle β between about 30° and about 90° may be used in the projecting position as this angle provides a beneficial hooking result. Auxiliary barb angles β larger than about 90° should however be avoided, as in this case the auxiliary barb 71 would lose its hooking function and the barb 31 could be pulled out. If the strength of the tissue is high and/or the pull force is high, it may even appear that the auxiliary barb 71 is pressed towards an auxiliary barb angle β of higher than about 120°. In this case the auxiliary barb 71 would lose its hooking effect.

In order to prevent this case, a deflection stopping member 73 may be provided at at least one of the barbs 31. The deflection stopping member 73 may be arranged below one or more auxiliary barbs 71 and provide a deflection stopping surface 75. The deflection stopping surface 75 projects from the barb 31 such that a deflection stopping angle γ is formed between the barb 31 and the deflection stopping surface 75. The deflection stopping angle γ of the deflection stopping surface 75 is determined on the basis of the desired maximum deflection angle of the auxiliary barb 71. In other words, if a pull force is applied to the barb 31 and the auxiliary barb 71 are pressed from the abutted position towards the projecting position, the maximum deflection of the auxiliary barb 71 is limited by the deflection stopping surface 75. In particular, the auxiliary barb 71 would be pressed against the deflection stopping surface 75, if the pull force is high and the auxiliary barb 71 would be deflected too extensively. The deflection stopping angle γ is measured between the insertion direction ID of the barb 31 and the deflection stopping surface 75.

As shown in FIG. 4, the deflection stopping member 73 may be provided at the barb point 69 of the barb 31 in the form of a thickened point. The upper surface of the thickened point, which faces the elongated body 67 of the suture material 9, may be the deflection stopping surface 75. The thickened point may have a tapered shape which tapers from the side which is closer to the elongated body 67 towards the point 77 of the barb 71.

The thickened point itself has additionally a hooking effect so that the removal of the barb 31 can be advantageously prevented.

Suture material 1 as described with respect to FIG. 4 may be beneficially used together with any one of the above described surgical apparatus 1.

The suture material 9 is arranged on the conveyor belt 7 of the surgical apparatus 1. By rotating the suture material dispensing member 21 in the first direction, the suture material is displaced towards the suture material application member 27. The suture material application member 27 is pressed onto a first portion of a tissue, whereby the barbs 31 are pressed into the tissue. In a next step, the surgical apparatus 1 is moved to another portion of the tissue and the suture material dispensing member 21 is further rotated in the first direction. The length of the dispensed suture material thereby defines the amount of the specified pull force which is applied to the suture material 9 and which is required so that the tissue portions are tied together in a preferred extent. By optionally rotating the suture material dispensing member 21 in the second direction, the length of the dispensed suture material 9 could be reduced or corrected, whereby the amount of the pull force could be increased. At the other portion of the tissue, the suture material application member 27 is again pressed onto the tissue so that one or more barbs 31 of the suture material 9 are pressed into the tissue. Thereby a suture method is enabled which allows suturing by only one instrument. This widens the spectrum of surgical techniques as suturing can be performed in body cavities that would be too narrow in space for conventional two instrument devices.

Moreover, by applying the pull force to the suture material 9, the auxiliary barbs 71 deflect towards the projecting position so that the barbs 31 are reliably hooked in the tissue.

Additionally, the surgical apparatus 1 may provide a suture material separation member 33 which separates the conveyor belt 7 from the suture material 9 in a specific portion. In this portion, a suture material cutting member 37 may be located, which is adapted to cut the suture material 9. As the suture material 9 is exposed in this portion, the suture material 9 can be easily cut by simply activating the suture material cutting member 37 after the specified (predetermined/predeterminable) pull force has been applied to the suture material 9. It is no longer necessary for the surgeon to use additional scissors for cutting the suture material.

1. A surgical apparatus for suturing tissue using a surgical suture material having one or more hooking elements, the surgical apparatus comprising:
a suture material transportation member adapted to transport the suture material;
a suture material dispensing member adapted to displace the suture material transportation member in a first direction for dispensing suture material to the tissue to be sutured and adapted to apply a specified pull force to the suture material; and
a suture material application member adapted to apply the suture material to the tissue so that at least the hooking elements are introduced into the tissue.

2. A surgical apparatus according to claim 1, wherein the suture material is adhered onto the suture material transportation member in order to be transported.

3. A surgical apparatus according to claim 1, wherein the suture material dispensing member is further adapted to displace the suture material transportation member.

4. A surgical apparatus according to claim 1, further comprising a suture material separation member adapted to separate the suture material from the suture material transportation member.

5. A surgical apparatus according to claim 4, further comprising a suture material cutting member adapted to cut the suture material in an area where the suture material is separated from the suture material transportation member by the suture material separation member.

6. A surgical apparatus according to claim 1, wherein the suture material transportation member is at least one of a conveyor belt, a conveyor chain, and a conveyor slide member.

7. A surgical apparatus according to claim 4, wherein the suture material separation member comprises at least one guide roller.

8. A surgical apparatus according to claim 5, wherein the suture material cutting member comprises at least one knife element or a heating wire.

9. A method for using a surgical apparatus comprising:
providing a surgical apparatus according to claim 1;
driving the suture material transportation member in the first direction using the suture material dispensing member so that the suture material is transported to the suture material application member and so that the specified pull force is applied to the suture material; and
introducing at least the hooking elements of the suture material (9) into the tissue by means of the suture material application member (27).

10. A method according to claim 9, further comprising driving the suture material transportation member in the second direction using the suture material dispensing member, wherein the suture material is transported by adhering the suture material onto the suture material transportation member.

11. A method according to claim 9, further comprising separating the suture material from the suture material transportation member using the suture material separation member.

12. A method according to claim 11, further comprising cutting the suture material at a position where the suture material is separated from the suture material transportation member by the suture material separation member.

13. A suture material comprising:
an elongated body;
a plurality of barbs attached to the elongated body; and
at least one auxiliary barb is attached to at least one of the plurality of barbs, wherein the auxiliary barb is directly or indirectly attached to the barb such that the auxiliary barb is abutted on the barb when the barb is inserted into the tissue and such that the auxiliary barb projects from the barb when a pull force is applied to the barb in a direction opposite to an insertion direction of the barb.

14. A suture material according to claim 13, wherein the barbs are attached perpendicular to the elongated body.

15. A suture material according to claim 13, wherein a deflection stopping member is attached to at least one of the barbs and adapted to stop a deflection of the auxiliary barb at a predetermined angle.

16. A surgical apparatus according to claim 2, wherein the suture material dispensing member is further adapted to displace the suture material transportation member.

17. A surgical apparatus according to claim 2, further comprising a suture material separation member adapted to separate the suture material from the suture material transportation member.

18. A surgical apparatus according to claim 3, further comprising a suture material separation member adapted to separate the suture material from the suture material transportation member.

19. A surgical apparatus according to claim 5, wherein the suture material separation member comprises at least one guide roller.

20. A surgical apparatus according to claim 6, wherein the suture material separation member comprises at least one guide roller.
